(19) United States
(12) Patent Application Publication
(10) Pub. No.: US 2022/0240887 A1
(43) Pub. Date: Aug. 4, 2022

(54) SYSTEM AND METHOD FOR OBTAINING AN X-RAY IMAGE OF A SUBJECT

(71) Applicant: ADAPTIX LTD, Begbroke (GB)

(72) Inventor: Conrad Dirckx, Begbroke (GB)

(21) Appl. No.: 17/718,677

(22) Filed: Apr. 12, 2022

Related U.S. Application Data
(63) Continuation of application No. PCT/GB2020/052539, filed on Oct. 12, 2020.

(30) Foreign Application Priority Data
Oct. 17, 2019 (GB) 1915036.6

(51) Int. Cl. A61B 6/00; A61B 6/02

(52) U.S. Cl. CPC A61B 6/547; A61B 6/025; A61B 6/587; A61B 6/4452; A61B 6/5205; A61B 6/4007

(57) ABSTRACT

Image quality for flat-panel array tomosynthesis acquisition is not equivalent to CT because of the reduced number of image angles that are acquired in DT, and also image quality is not uniform throughout the volume and decreases near to the fixed detector array and the fixed flat-panel source. The present invention provides a system for obtaining an X-ray image of a subject 1 in which an emitter panel 3 and detector 7 are movable on a smart armature 9. In this way, image quality can be improved, for example by combining acquisitions taken from different orientations, thereby approaching the quality of conventional CT imaging, whilst still maintaining the lower cost and dose of conventional DT imaging.

### RELATED APPLICATIONS

This application claims the benefit under 35 U.S.C. § 120, and is a continuation, of co-pending International Application PCT/GB2020/052539, filed Oct. 12, 2020 and designating the US, which claims priority to GB Application 1915036.6, filed Oct. 17, 2019, such GB Application also being claimed priority to under 35 U.S.C. § 119. These GB and International applications are incorporated by reference herein in their entireties.

### FIELD

The present invention relates generally to a system and method for obtaining an X-ray image of a subject and finds particular, although not exclusive, utility in digital tomosynthesis.

### BACKGROUND

Conventional x-ray machines produce a single 2D image by illuminating a region of interest from a single point source, and projecting onto a flat detector. Complex 3D anatomical shapes are therefore difficult to represent well; however, the system is relatively cheap and subjects receive only a relative low dose.

C-arm x-ray machines use a conventional x-ray source on a mechanical C-shaped arm where source and detector are in a fixed position relative to each other & moved together around the subject.

3D medical imaging using x-rays has been possible since the invention of tomosynthesis using a conventional x-ray point source that is moved through a range of angles in a single direction. A limited angular range of the images required leads to “tomosynthesis artefact”—i.e. bleeding of image features between slices, non-isotropic voxel sizes and reduced image quality out of the plane of focus.

Computed tomography (CT) imaging in the 1970s remains today the gold standard for 3D x-ray imaging, offering fast acquisition & reconstruction speeds and excellent image quality. This uses a single x-ray sources coupled with detector arrays that are mechanically moved 360 degrees around the volume of interest. Due to the fixed emission cone profile from the single x-ray source, the location of the detector array is determined by the location of the x-ray source; that is, the relative displacement of the detector array from the x-ray source doesn't change, specifically, the source and detector are moved together in an arc around the volume of interest.

### SUMMARY

In recent years, x-ray digital tomosynthesis (DT) techniques have been developed which offer the possibility of 3D image reconstruction at lower cost and dose, by using static arrays of x-ray sources to perform tomosynthesis acquisitions without the need to move the source or detectors. However, image quality for flat-panel array tomosynthesis acquisition is not equivalent to CT because of the reduced number of image angles that are acquired in DT, and also image quality is not uniform throughout the volume and decreases near to the fixed detector array and the fixed flat-panel source.

According to a first aspect of the present invention, there is provided a system for obtaining an X-ray image of a subject, the system comprising: an X-ray emitter panel; an X-ray detector; an armature for moving the emitter panel and detector; at least one sensor for determining a spatial position of the emitter panel and the detector; a spatial tracker for monitoring the position of the detector and the emitter panel, in response to receiving a spatial position signal from the at least one sensor.

In this way, image quality can be improved, approaching that of conventional CT imaging, whilst still maintaining the lower cost and dose of conventional DT imaging.

The system may comprise a DT system. The subject may comprise a person, animal, piece of equipment, inanimate object and/or a part thereof.

The X-ray emitter panel may comprise an array of x-ray emitters, which may be individually addressable. The emitter panel may be substantially flat, but in alternative arrangements could be curved. The emitter panel may have a substantially square or rectangular form, but in alternative arrangements other configurations are envisaged such as hexagonal. The emitters may be arranged in a square or triangular arrangement on the panel.

The X-ray detector panel may comprise an array of pixels. The detector panel may be substantially flat, but in alternative arrangements could be curved. The detector panel may have a substantially square or rectangular form, but in alternative arrangements other configurations are envisaged such as hexagonal. The pixels may be arranged in a square or triangular arrangement on the panel.

The emitter panel may be located on a first armature. The detector may comprise a detector panel and may be located on a second armature. The or each panel being located on a respective armature may comprise the or each panel being connected and/or coupled to the respective armature.

The armature (and/or first and/or second armature) being movable may comprise having one, two, three, four, five or more degrees of freedom, such that the respective panel may be moved to substantially any position within a region, and/or may be orientated to substantially any orientation. For example, the first and/or second armature may allow translation of the respective panel in substantially three Cartesian directions (i.e. in three dimensions) between a maximum and a minimum respective extension, and/or the first and/or second armature may permit rotation of the panel about first and/or second axes with respect to some external reference frame.

The armature and/or first and/or second armature may be jointed and/or telescopic or otherwise extensible. In cases where the armature(s) is jointed, one or more members on opposing sides of one or more joints may be telescopic or otherwise extensible; alternatively, all members may be fixed (i.e. non-telescopic/extensible). One or more joints may permit one or more axes of rotation; for example, the joints may be hinge joints, planar joints, ball joints, or similar. The armature(s) may be flexible and/or malleable, or may be resilient; for instance, comprising unitary joint-members (not distinct joints from the members).

Determining a spatial position of the emitter and/or detector panel may comprise determining the location and/or orientation of the emitter and/or detector panel.

The at least one sensor may comprise at least one first sensor for determining a spatial position of the emitter panel, and at least one second sensor for determining a spatial position of the detector. The at least one sensor, and/or the at least one first and/or second sensor, may comprise only one or a plurality of first and/or second sensors. The sensor(s) may determine a spatial position of the respective panel directly (for example by determining the location of reference pin(s), transceiver(s), etc. located on the panel) or may infer the spatial position of the respective panel by determining an amount of extension of the or each member, and/or determining an amount of rotation of the or each joint. For example, a respective sensor may be located at each pivot point.

The first and second armatures may be independent of one another. Alternatively or additionally, the system may comprise a master armature permitting large-scale movement of the emitter and detector panels together, and then small-scale movement of the emitter and/or detector panels relative to one another by virtue of the first and/or second armatures. For example, the first or second armature may comprise the master armature, and the second or first armature (respectively) may be coupled to the first armature. Alternatively, the first and second armatures may both be coupled to the master armature.

The armature(s) may comprise smart armature(s). The sensor(s) may be configured to send respective spatial position signals to the spatial tracker.

The spatial tracker may comprise a processor, and may be embodied in a computer system.

The position of the detector panel and the emitter panel may comprise the relative position of the detector panel relative to the emitter panel and/or the relative position of the emitter panel relative to the detector panel. The position of the detector panel and the emitter panel may comprise the absolute position of the detector panel and the emitter panel, or the position of the detector panel and the emitter panel relative to some predefined/predetermined reference point.

The spatial tracker may be configured to determine a separation of the emitter and detector panels.

The separation of the emitter and detector panels may comprise a shortest, longest and/or average distance from the detector panel to the emitter panel in a direction normal to the detector and/or emitter panel. The separation of the emitter and detector panels may comprise a shortest, longest and/or average distance from at least one predefined/predetermined point on the detector panel to at least one predefined/predetermined point on the emitter panel in substantially any direction. The separation of the emitter and detector panels may comprise a shortest, longest and/or average path length taken by x-rays emitted from the emitter panel to reach the detector panel. However, other determinations of the separation are also envisaged.

Alternatively, the separation of the emitter and detector panels may comprise the spatial location of the emitter and/or detector panels (or at least one location thereon) with respect to an external reference frame and/or some predefined/predetermined position.

The spatial tracker may be configured to determine an orientation of the detector panel with respect to the emitter panel.

The orientation of the detector panel may comprise the inclination of one panel relative to the other panel, and/or the direction of that inclination. Alternatively, the orientation of the detector panel with respect to the emitter panel may comprise determining the orientation of the detector panel and/or the orientation of the emitter panel with respect to a predetermined/predefined reference orientation.

The spatial tracker may be configured to determine a lateral displacement of the detector panel from an axis of the emitter panel.

The lateral displacement of the detector panel may comprise overlap of x-rays emitted from the emitter panel onto the detector panel. The lateral displacement of the detector panel may comprise a spacing between only one or at least one predefined/predetermined point on one panel and only one or at least one predefined/predetermined point on another panel in a direction parallel to the one panel and spaced from the one panel by the separation distance of the panels.

The spatial tracker may be configured to determine an absolute position of the emitter and detector panels. That is, the spatial tracker may determine the position of the emitter and detector panels relative to a fixed point.

The absolute position of the emitter and detector panels may comprise the absolute location and/or the absolute orientation of the panels.

The spatial tracker may be configured to determine the position of the emitter and detector panels relative to a previous position of the emitter and detector panels. In this way, imaging may be carried out from two or more sides of an object of interest.

A predefined/predetermined point on one of the panels may comprise the center, a corner, or some other location on the panel.

The system may further comprise a reconstruction processor for reconstructing a 3D image of a region between the emitter and detector panels.

According to a second aspect of the present invention, there is provided a method of obtaining an X-ray image of a subject, the method comprising the steps of: providing an X-ray emitter panel; providing an X-ray detector; moving the X-ray emitter panel to a first location using an armature; determining a spatial position of the emitter panel with at least one sensor; moving the X-ray detector panel to a second location using the armature; determining a spatial position of the detector panel with the at least one sensor; monitoring the position of the detector panel and the emitter panel with a spatial tracker, in response to receiving a spatial position signal from the at least one sensor; and acquiring imaging data by activating emitters on the emitter panel.

A controller (e.g. computer controller) may determine from the position of the detector panel and the emitter panel whether x-rays emitted from each emitter may impinge the detector, and the controller may only activate emitters which will impinge the detector during image data acquisition. In this way, unnecessary radiation dose to a subject can be avoided.

In addition, the acquired image data may be used to identify a region of interest for subsequent acquisition from a new emitter and detector position (e.g. by creating a conventional/composite 2D image). This may be done manually or automatically.

In some embodiments, acquired image data from a first acquisition may be used to identify a region of interest for subsequent acquisition from the same emitter and detector positions. This may be done manually or automatically.

Automatic medical image analysis and feature detection algorithms are in increasingly widespread use, particularly for CT. This would be in applications such as automatic view preparation, automatic spine labelling etc. as well as more advanced CAD applications to be used in screening. In digital tomosynthesis, there is some existing automatic feature detection work available such as automatic nipple detection in mammography (see “Fully automated nipple detection in digital breast tomosynthesis”, Computer Methods and Programs in Biomedicine, Volume 143 Issue C, May 2017 Pages 113-120).

In fact, such a 2D image may be used to identify an optimal new emitter and detector position for image data acquisition of the region of interest. An indication of the new emitter and detector position may be sent back to the spatial tracker either to automatically move the armature(s) (for instance with servos or motors) or to provide instructions to an operator for manual manipulation of the armature(s).

In any event, the armature(s) may be positioned manually by an operator for a first acquisition of image data.

The method may further comprise the step of reconstructing a 3D image of a region between the emitter panel and detector panel with a processor using the acquired imaging data.

In this way, the 3D image may be used to identify a region of interest for subsequent acquisition from a new emitter and detector position. In fact, the 3D image may be used to identify an optimal new emitter and detector position for image data acquisition of the region of interest, as above.

The user may manually select the region of interest using 2D or 3D image display software or through automatic image feature identification—for example through automatic spine detection. Region of interest identification allows for dose and acquisition time to be minimized without compromising image quality by reducing the number of emitters that are used during the second acquisition.

The method may further comprise the steps of: moving the X-ray emitter panel to a third location; determining a new spatial position of the emitter panel with the at least one sensor; moving the X-ray detector panel to a fourth location; determining a new spatial position of the detector with the at least one sensor; monitoring the new position of the detector panel and the emitter panel with a spatial tracker, in response to receiving a new spatial position signal from the at least one sensor; and acquiring further imaging data by activating emitters on the emitter panel.

The emitter and detector may be moved, for example by rotating by a desired angle (typically 90 degrees). One version of this device could automate the positioning and select the optimal position for the second acquisition based on the calculation of the optimal sampling scheme based on the angles from the first scan and the selected region/volume of interest.

The controller may determine from the new position of the detector panel and the emitter panel whether x-rays emitted from each emitter may impinge the detector, and the controller may only activate emitters which will impinge the detector during image data acquisition. Similarly, the controller may determine from the new position of the detector panel and the emitter panel, and/or the region of interest, whether x-rays emitted from each emitter may impinge the detector and/or pass through the region of interest, and the controller may only activate emitters which will impinge the detector and/or pass through the region of interest during image data acquisition.

During the second acquisition, the set of emitters used will be further constrained by restricting the acquisition to those emitters that will overlap with the selected volume of interest. The effect of this is to both reduce the acquisition time and also to limit the total dose that is delivered to the patient.

The method may further comprise the step of reconstructing a 3D image of a region between the emitter panel and detector panel with a processor using the acquired imaging data and the further imaging data.

That is, combining images from first and second data sets to form a composite data set of a region of interest. This significantly improves the image quality provided by flat-panel array tomosynthesis by allowing 2 or more sets of acquisition data to be combined to provide a 3D dataset with higher image quality. In some sense, this adds the positioning flexibility of a C-arm system with the benefits of a flat panel tomosynthesis system.

During the reconstruction of the 2nd reconstruction, raw frame data along with the positional information is used from both datasets and are used to form the single 3D dataset.

Further image data acquisitions may be made to improve a final reconstruction, for instance in view of a further limitation to the region of interest, and/or by providing different angles of emission and detection.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a representation of a system for obtaining an X-ray image of a subject.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other. For instance, wireless connectivity is contemplated.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

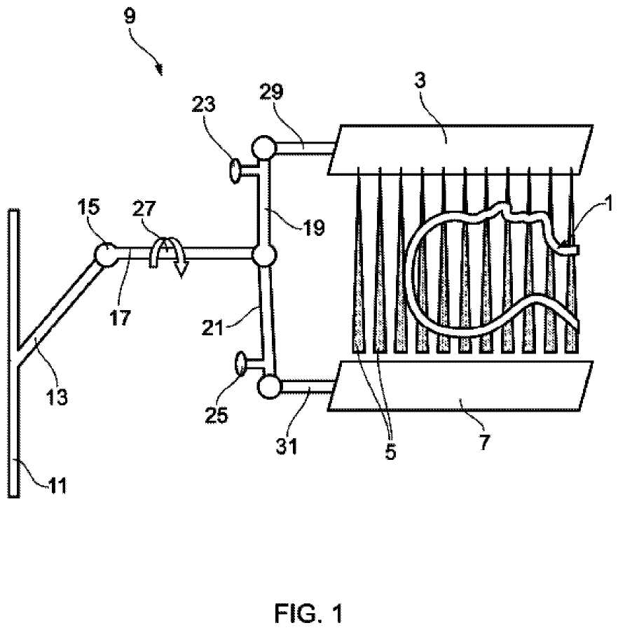

FIG. 1 is a representation of a system for obtaining an X-ray image of a subject 1, which includes an emitter panel 3 configured to emit individual cones of x-rays 5 from a plurality of respective x-ray emitters. For clarity only a subset of non-overlapping x-ray cones 5 are shown. Each cone of x-rays passes through a region between the emitter panel and detector 7, some of which pass through the subject 1, to impinge on the detector 7.

The emitter panel 3 may comprise a 20×20 cm flat panel addressable x-ray source, but other sizes and configurations are envisaged. Similarly, the detector 7 may comprise a 30×30 cm dynamic detector, but again, other sizes and configurations are envisaged.

The emitter panel 3 and the detector 7 are each mounted on an armature structure 9, which is secured at an opposing end to a fixed reference point (e.g. wall or stanchion) 11. Armature structure 9 comprises a master armature comprising a fixed member 13 connected about rotating joint 15 to a rotating member 17. From the free end of rotating member 17 are attached a first telescopic armature 19 and a second telescopic armature 21 opposing the first telescopic armature 19. Handles 23, 25 on the telescopic armatures 19, 21 permit manual extension and retraction of the telescopic armatures, as well as rotation 27 about the rotating joint 15. Each telescopic armature 19, 21 is attached at its opposing end to a respective one of the emitter panel 3 and detector 7 by connecting members 29, 31.

This allows for at least 90 degrees rotation of source/detector pair and for increasing/reducing the distance between the source detector between 20 cm and 50 cm. Typically, the source and detector will be parallel to one another (as shown) but in alternative arrangements they can be positioned independently in situations where the detector position cannot easily be changed.

Positional sensors (not shown) are included which determine the relative position of the source & detector to within one 1 cm, in particular to within 5 mm, more particularly to within 1 mm.

Also not shown are a control and reconstruction computer running software that is capable of reconstruction of a standard tomosynthesis dataset using a set of 2D frames, and displaying a 3D reconstructed image in a GUI which contains a Volume of Interest drawing tool that allows a user to highlight a volume of interest.

Optionally the control and reconstruction computer may determine the emitters that should be used based on the source and detector position, such that cones 5 that will not impinge the detector 7 are not activated.

Similarly, the control and reconstruction computer may be configured to calculating the emitters that should be used in a second acquisition based on the identified Volume of Interest, together with source and detector position.

As a further alternative, the control and reconstruction computer may be configured to calculate an optimal position for the source and detector for a second acquisition based on the Volume of Interest, reconstructed volume and angular information from the first scan.

Furthermore, the control and reconstruction computer may be configured for reconstruction of a tomosynthesis dataset using 2 sets of orthogonal input datasets, or potentially two input datasets of arbitrary relative position.

For example, to optimally acquire 3D images of the cervical spine, one dataset would be acquired in the anterior-posterior direction of the head and neck and reconstructed to form a 3D volume. Automatic or manual region identification techniques would be used to identify a volume of interest around the cervical spine. A second set acquired orthogonally in the Mediolateral direction with only emitters that intercept the region of interest would be acquired. A full 3D reconstruction could then be performed using both datasets.

1. A system for obtaining an X-ray image of a subject, the system comprising:
an X-ray emitter panel comprising an array of X-ray emitters;
an X-ray detector;
an armature for moving the emitter panel and detector;
at least one sensor for determining a spatial position of the emitter panel and the detector;
a spatial tracker for monitoring the position of the detector and the emitter panel, in response to receiving a spatial position signal from the at least one sensor; and
a controller configured to activate a first set of emitters of the emitter panel to identify a region of interest, the controller further configured to activate a second set of emitters of the emitter panel, the second set of emitters restricted to be only those emitters of the emitter panel that will overlap with the region of interest.

2. The system of claim 1, wherein the spatial tracker is configured to determine a separation of the emitter panel and detector.

3. The system of claim 1, wherein the spatial tracker is configured to determine an orientation of the detector with respect to the emitter panel.

4. The system of claim 1, wherein the spatial tracker is configured to determine a lateral displacement of the detector from an axis of the emitter panel.

5. The system of claim 1, wherein the spatial tracker is configured to determine an absolute position of the emitter panel and detector.

6. A method of obtaining an X-ray image of a subject, the method comprising the steps of:
providing an X-ray emitter panel comprising an array of X-ray emitters;
providing an X-ray detector;
providing a controller;
moving the X-ray emitter panel to a first location using an armature;
determining a spatial position of the emitter panel with at least one sensor;
moving the X-ray detector to a second location using the armature;
determining a spatial position of the detector with the at least one sensor;
monitoring the position of the detector and the emitter panel with a spatial tracker, in response to receiving a spatial position signal from the at least one sensor; and
acquiring imaging data by activating emitters on the emitter panel, by using the controller to activate a first set of emitters of the emitter panel to identify a region of interest, and to activate a second set of emitters of the emitter panel, the second set of emitters restricted to be only those emitters of the emitter panel that will overlap with the region of interest.

7. The method of claim 6, further comprising the step of reconstructing a 3D image of a region between the emitter panel and detector with a processor using the acquired imaging data.

8. The method of claim 6, further comprising the steps of:
moving the X-ray emitter panel to a third location;
determining a new spatial position of the emitter panel with the at least one sensor;
moving the X-ray detector to a fourth location;
determining a new spatial position of the detector with the at least one sensor;
monitoring the new position of the detector and the emitter panel with a spatial tracker, in response to receiving a new spatial position signal from the at least one sensor; and
acquiring further imaging data by activating emitters on the emitter panel.

9. The method of claim 8 further comprising the step of reconstructing a 3D image of a region between the emitter panel and detector with a processor using the acquired imaging data and the further imaging data.
